(19) United States
(12) Patent Application Publication
(10) Pub. No.: US 2025/0261673 A1
(43) Pub. Date: Aug. 21, 2025

(54) NATURAL LACTONES AND HYDROXY FATTY ACIDS AND METHODS OF MAKING SAME

(71) Applicant: INTERNATIONAL FLAVORS & FRAGRANCES, INC., NEW YORK, NY (US)

(72) Inventors: Hsi-Wen Chin, Tinton Falls, NJ (US); Fuyuan Jing, Morganville, NJ (US)

(21) Appl. No.: 18/245,022

(22) PCT Filed: Sep. 7, 2021
(86) PCT No.: PCT/US2021/049223

§ 371 Date: Mar. 13, 2023

Related U.S. Application Data
(60) Provisional application No. 63/079,740, filed on Sep. 17, 2020.

(51) Int. Cl. A23L 27/24; A23L 33/115; A23L 33/135; C12N 1/16; C12N 1/20; C12P 7/6409; C12P 17/04

(52) U.S. Cl. CPC A23L 27/24; A23L 33/115; A23L 33/135; C12N 1/16; C12N 1/20; C12P 7/6409; C12P 17/04

(57) ABSTRACT

Fermented extracts containing natural lactones and hydroxy fatty acids are extracted from mixed culture fermentations of non-hydrolyzed vegetable oils.

### CROSS REFERENCE

This application is a 371 of International Application No. PCT/US2021/049223 filed Sep. 7, 2021 which claims the benefit of U.S. Provisional Application No. 63/079,740 filed Sep. 17, 2020 which are incorporated by reference in their entirety.

### FIELD OF THE INVENTION

Fermented extracts including natural lactones and hydroxy fatty acids along with fermentation methods of making natural lactones and hydroxy fatty acids.

### BACKGROUND OF THE INVENTION

Today's food product consumers are increasingly interested in natural ingredients, but they are unwilling to compromise the taste qualities of those food products. They expect natural food products and the natural ingredients in those food products to have rich and satisfying taste profiles. Taste compounds that contribute to providing such rich and satisfying taste profiles include lactones and hydroxy fatty acids. For example, gamma-dodecalactone is found in apricot, peach, pineapple, and cheddar cheese and can provide fatty, fruity, milky, and peach-like flavors. Gamma-dodecen-6-lactone has a powerful dairy note with a sweet, fatty, creamy, waxy, and fruity odor. To date, these taste compounds have been synthetically produced or have been produced using complicated multi-step fermentation processes or fermentation processes involving pathogenic microorganisms.

In EP0578388B1, International Flavors & Fragrances Inc. describes a fermentation process using Pseudomonas bacteria to act on triglycerides to generate hydroxy carboxylic acids that are then fermented with yeast to generate lactones.

In JP2003250594, Ogawa and Co describe a microbial manufacture of cis-6-dodecen-4-olide from linoleic acid in which the microbes include lactic acid bacteria and beta-oxidizing yeast. A necessary first step before the microbial fermentation can begin is the hydrolysis of fat to generate linoleic acid. This preparatory step increases the complexity of the process.

Thus, there is a need for natural lactones and hydroxy fatty acids produced from food-grade microorganisms using simpler fermentation processes.

### SUMMARY OF THE INVENTION

Our inventions include fermented extracts that are extracted from a mixed culture fermentation of a non-hydrolyzed vegetable oil. The mixed culture fermentation can include at least one bacterium and at least one yeast. Advantageously, the at least one bacterium includes Lactobacillus lactis, Lactobacillus planatarum, Lactobacillus casei, Lactobacillus acidophilus, or combinations thereof. Further, the at least one yeast can include Yarrowia lipolytica, Candida petrophilum, or combinations thereof. In preferred embodiments, the fermented extract comprises from about 10% to about 50% w/w gamma-dodecen-6-lactone and from about 5% to about 25% w/w gamma-dodecalactone.

Our inventions also include methods of making taste compounds comprising the steps of:
a. Co-culturing at least one non-pathogenic lactic acid bacterium and at least one yeast to create a mixed culture;
b. fermenting a non-hydrolyzed vegetable oil in the mixed culture to create a mixed culture fermentation medium;
c. acidifying the mixed culture fermentation medium by admixing an acid following the fermentation step to create an acidified mixed culture fermentation medium; and
d. lactonizing the acidified mixed culture fermentation medium by heating the acidified mixed culture fermentation medium; wherein the method generates at least one of a gamma lactone, an hydroxy fatty acid, or combinations thereof.

These methods additionally can include preparatory steps such as preparing a seed culture of either the at least one non-pathogenic lactic acid bacterium, the yeast or both. The co-culturing step can also include first culturing the at least one non-pathogenic lactic acid bacterium by adding the bacterial seed culture to a bacterial fermentation medium and then adding the at least one yeast seed culture to create the mixed culture. In some embodiments of these inventive methods, the non-hydrolyzed vegetable oil includes sunflower oil, safflower oil, or combinations of those oils.

In addition to preparatory steps, the inventive methods can include down-stream processing steps such as solvent extraction following lactonization and a distillation step following the solvent extraction step can create a refined lactones product. The output of the method can include gamma-dodecen-6-lactone, gamma-dodecalactone, or combinations of those gamma lactones. The output can also include hydroxy fatty acids such as stearic acid, oleic acid, linoleic acid, or combinations of those hydroxy fatty acids.

Further, our inventions include products comprising a fermented extract and a product base wherein the fermented extract is extracted from a mixed culture fermentation of a non-hydrolyzed vegetable oil. For these inventions, the fermented extract can be included at an olfactory effective amount. In some of these inventions, the olfactory effective amount is from about 10 ppb to about 1000 ppm.

### OVERVIEW

1. A fermentation medium comprising at least one bacterium, at least one yeast, and at least one non-hydrolyzed vegetable oil.
2. The fermentation medium as in claim 1, wherein the at least one bacterium is capable of producing 18-carbon 10-hydroxy fatty acids.
3. The fermentation medium as in claim 1, wherein the at least one bacterium includes a non-pathogenic lactic acid bacterium.
4. The fermentation medium as in claim 1, wherein the at least one bacterium includes Lactobacillus lactis, Lactobacillus plantarum, Lactobacillus casei, Lactobacillus acidophilus, or combinations thereof.
5. The fermentation medium as in claim 1, wherein the at least one yeast is capable of oxidizing 18-carbon 10-hydroxy fatty acids to produce 12-carbon 4-hydroxy fatty acids.
6. The fermentation medium as in claim 1, wherein the at least one yeast includes Yarrowia, Saccharomyces, Candida or combinations thereof.
7. The fermentation medium as in claim 1, wherein the at least one yeast includes Yarrowia lipolytica, Candida petrophilum, or combinations thereof.
8. The fermentation medium as in claim 1, wherein the non-hydrolyzed vegetable oil includes sunflower oil, safflower oil, or combinations thereof.
9. The fermentation medium as in claim 1, wherein the non-hydrolyzed vegetable oil contains at least 70% unsaturated fatty acids.
10. The fermentation medium as in claim 1, wherein the non-hydrolyzed vegetable oil has at least 75% linoleic acid.
11. A fermented extract comprising from about 50% to about 95% w/w gamma-dodecen-6-lactone and from about 10% to about 50% w/w gamma-dodecalactone.
12. The fermented extract as in claim 11, further comprising at least one hydroxy fatty acid.
13. The fermented extract as in claim 11, further comprising at least one free fatty acid.
14. The fermented extract as in claim 11, wherein the fermented extract has a ratio of gamma-dodecen-6-lactone to gamma-dodecalactone of from about 2:1 to about 4:1.
15. A fermented extract extracted from a mixed culture fermentation of a non-hydrolyzed vegetable oil.
16. The composition as in claim 15, wherein the mixed culture fermentation comprises at least one bacterium and at least one yeast.
17. The composition as in claim 15, wherein the at least one bacterium is capable of producing 18-carbon 10-hydroxy fatty acids.
18. The composition as in claim 16, wherein the at least one bacterium includes a non-pathogenic lactic acid bacterium.
19. The composition as in claim 16, wherein the at least one bacterium includes Lactobacillus lactis, Lactobacillus planatarum, Lactobacillus casei, Lactobacillus acidophilus, or combinations thereof.
20. The composition as in claim 15, wherein the at least one yeast is capable of oxidizing 18-carbon 10-hydroxy fatty acids to produce 12-carbon 4-hydroxy fatty acids.
21. The composition as in claim 15, wherein the at least one yeast includes Yarrowia, Saccharomyces, Candida or combinations thereof.
22. The composition as in claim 16, wherein the at least one yeast includes Yarrowia lipolytica, Candida petrophilum, or combinations thereof.
23. The composition as in claim 15, wherein the non-hydrolyzed vegetable oil includes sunflower oil, safflower oil, or combinations thereof.
24. The composition as in claim 15, wherein the non-hydrolyzed vegetable oil contains at least 70% unsaturated fatty acids.
25. The composition as in claim 15, wherein the non-hydrolyzed vegetable oil has at least 75% linoleic acid.
26. The composition as in claim 15, further comprising at least one 18-carbon 10-hydroxy fatty acid, at least one 12-carbon gamma-lactone, or combinations thereof.
27. The composition as in claim 15, wherein the fermented extract comprises from about 10% to about 50% w/w gamma-dodecen-6-lactone and from about 5% to about 25% w/w gamma-dodecalactone.
28. The composition as in claim 27, wherein the fermented extract has a ratio of gamma-dodecen-6-lactone to gamma-dodecalactone of from about 2:1 to about 4:1.
29. A method of making taste compounds comprising the steps of:
   a. co-culturing at least one non-pathogenic lactic acid bacterium and at least one yeast to create a mixed culture;
   b. fermenting a non-hydrolyzed vegetable oil in the mixed culture to create a mixed culture fermentation medium;
   c. acidifying the mixed culture fermentation medium by admixing an acid following the fermentation step to create an acidified mixed culture fermentation medium; and
   d. lactonizing the acidified mixed culture fermentation medium by heating the acidified mixed culture fermentation medium; wherein the method generates at least one of a gamma lactone, an hydroxy fatty acid, or combinations thereof.
30. The method as in claim 29, wherein the lactonization step has a lactonization temperature of from about 90 C to about 130 C.
31. The method as in claim 29, wherein the lactonization step has a lactonization time of from about 10 minutes to about 60 minutes.
32. The method as in claim 29, further comprising at least one step of preparing a seed culture of the at least one non-pathogenic lactic acid bacterium, preparing a seed culture of the at least one yeast prior to the co-culturing step, or combinations thereof.
33. The method as in claim 32, wherein the co-culturing step further comprises first culturing the at least one non-pathogenic lactic acid bacterium by adding the at least one non-pathogenic lactic acid bacterium seed culture to a bacterial fermentation medium to generate an at least one non-pathogenic lactic acid bacterium culture.
34. The method as in claim 33, further comprising adding the at least one yeast seed culture to the at least one non-pathogenic lactic acid bacterium culture to create the mixed culture.
35. The method as in claim 29, wherein the non-hydrolyzed vegetable oil includes sunflower oil, safflower oil, or combinations thereof.
36. The method as in claim 29, wherein the non-hydrolyzed vegetable oil contains at least 70% unsaturated fatty acids.
37. The method as in claim 29, wherein the non-hydrolyzed vegetable oil has at least 75% linoleic acid.
38. The method as in claim 29, wherein the non-hydrolyzed vegetable oil has less than 2% free fatty acids.
39. The method as in claim 29, wherein the fermentation step has a fermentation time of from about 30 hours to about 200 hours.
40. The method as in claim 29, wherein the fermentation step has a fermentation temperature of from about 20 C to about 37 C.
41. The method as in claim 29, wherein the fermentation step has a fermentation pH of from about 4 to about 8.
42. The method as in claim 29, wherein the fermentation step has a fermentation dissolved oxygen content of less than about 35%.
43. The method as in claim 29, wherein the acidified mixed culture fermentation medium has an acidification pH from about 3 to about 4.
44. The method as in claim 29, wherein the acid includes hydrochloric acid, nitric acid, phosphoric acid, sulfuric acid, citric acid, or combinations thereof.
45. The method as in claim 29, further comprising a solvent extraction step following lactonization to create a fermented extract.
46. The method as in claim 45, further comprising a distillation step following the solvent extraction to create a refined lactones product.
47. The method as in claim 45, wherein the solvent extraction step involves a solvent including ethanol, ethyl acetate, or combinations thereof.
48. The method as in claim 46, wherein the refined lactones product includes at least 70% gamma-dodecen-6-lactone.
49. The method as in claim 46, wherein the refined lactones product has from about 5% to about 30% gamma-dodecalactone w/w by weight of the refined lactones product.
50. The method as in claim 29, wherein the at least one gamma lactone includes gamma-dodecen-6-lactone, gamma-dodecalactone, or combinations thereof.
51. The method as in claim 29, wherein the at least one hydroxy fatty acid includes stearic acid, oleic acid, linoleic acid, or combinations thereof.
52. The method as in claim 29, wherein the at least one non-pathogenic lactic acid bacterium includes Lactobacillus lactis, Lactobacillus planatarum, Lactobacillus casei, Lactobacillus acidophilus, or combinations thereof.
53. The method as in claim 29, wherein the at least one yeast includes Yarrowia lipolytica, Candida petrophilum, or combinations thereof.
54. A taste compound made by the process as in claim 29.
55. A method of modifying a product base comprising the steps of adding an olfactory effective amount of a fermented extract extracted from a mixed culture fermentation of non-hydrolyzed vegetable oil to a product base to create a modified product.
56. The method as in claim 55, wherein the product base includes at least one of a human food, an animal food, a dietary supplement, a medicament, a galenical composition, a cosmetic composition, a fragrance composition, a perfumed article, a pharmaceutical composition, or combinations thereof.
57. The method as in claim 55, wherein the product base includes at least one of a dairy product, a meat product, a plant-based protein product, or combinations thereof.
58. The method as in claim 55, wherein the olfactory effective amount is from about 10 ppb to about 200 ppm.
59. The method as in claim 55, wherein the modified product has a decreased perception of off-notes when compared to the product base as measured by sensory testing.
60. The method as in claim 55, wherein the modified product has an increased perception of dairy notes when compared to the product base as measured by sensory testing.
61. The method as in claim 55, wherein the modified product has an increased perception of at least one sensory attribute when compared to the product base as measured by sensory testing.
62. The method as in claim 61, wherein the at least one sensory attribute includes creamy, milky, fatty, waxy, rich, sweet, peachy, lactonic, fruity, or combinations.
63. A product comprising a fermented extract and a product base wherein the fermented extract is extracted from a mixed culture fermentation of non-hydrolyzed vegetable oil.
64. The product as in claim 63, wherein the product base includes at least one of a human food, an animal food, a dietary supplement, a medicament, a galenical composition, a cosmetic composition, a fragrance composition, a perfumed article, a pharmaceutical composition, or combinations thereof.
65. The product as in claim 63, wherein the product base includes at least one of a dairy product, a meat product, a plant-based protein product, or combinations thereof.
66. The product as in claim 63, wherein the fermented extract comprises from about 10% to about 50% w/w gamma-dodecen-6-lactone and from about 5% to about 25% w/w gamma-dodecalactone.
67. The product as in claim 63, wherein the fermented extract is a refined lactones product comprising 50% to about 95% w/w gamma-dodecen-6-lactone and from about 5% to about 50% w/w gamma-dodecalactone.
68. The product as in claim 67, wherein the refined lactones product has a ratio of gamma-dodecen-6-lactone to gamma-dodecalactone of from about 2:1 to about 4:1.
69. The product as in claim 67, wherein the refined lactones product is present in an olfactory effective amount.
70. The product as in claim 67, wherein the refined lactones product is present in an amount of from about 10 ppb to about 200 ppm.
71. The product as in claim 63, wherein the fermented extract is present in an olfactory effective amount.
72. The product as in claim 63, wherein the fermented extract is present in an amount of from about 10 ppb to about 1000 ppm.
73. The product as in claim 63, wherein the product has a decreased perception of off-notes when compared to the product base as measured by sensory testing.
74. The product as in claim 63, wherein the product has an increased perception of dairy notes when compared to the product base as measured by sensory testing.
75. The product as in claim 63, wherein the product has an increased perception of at least one sensory attribute when compared to the product base as measured by sensory testing.
76. The product as in claim 75, wherein the at least one sensory attribute comprises creamy, fatty, rich, sweet, milky, waxy, lactonic, peachy, fruity, or combinations thereof.

### DETAILED DESCRIPTION OF THE INVENTION

To address the need for generating natural lactones and hydroxy fatty acids by using food-grade microorganisms in a simple fermentation process, these inventors have developed fermentation media, fermented extracts, and fermentation processes using mixed cultures of food-grade, non-pathogenic lactic acid bacteria and yeast acting on non-hydrolyzed vegetable oils. The natural lactones and hydroxy fatty acids in these compositions and processes have been found beneficial in imparting an olfactory effect taste enhancement and/or somatosensory effect to various consumable products.

In some embodiments, a method of making taste compounds includes the steps of:
i. co-culturing at least one non-pathogenic lactic acid bacteria and at least one yeast to create a mixed culture;
ii. fermenting a non-hydrolyzed vegetable oil in the mixed culture to create a mixed culture fermentation medium;
iii. acidifying the mixed culture fermentation medium by admixing an acid following the fermentation step to create an acidified mixed culture fermentation medium, and
iv. lactonizing the acidified mixed culture fermentation medium; wherein the method generates at least one gamma lactone, at least one hydroxy fatty acid, or combinations thereof.

As used herein “co-culturing” is understood to mean a fermentation process in which the fermentation medium contains two or more organisms. This mixed culture fermentation medium can also be called a consortium culture, and the process can be called a consortium fermentation.

The chemical reactions shown below illustrate the overall process and show how the non-hydrolyzed vegetable oil undergoes biotransformation due to the consortia fermentation to generate desirable taste compounds.

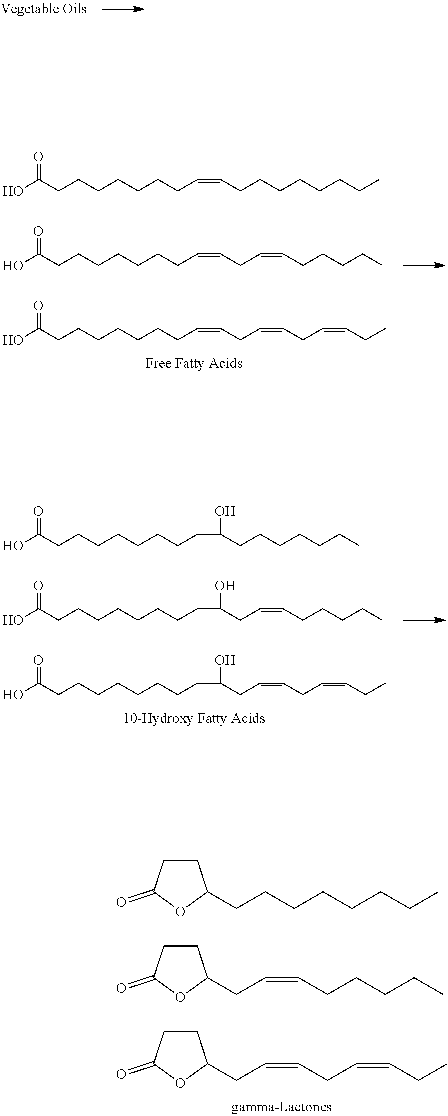

The consortia fermentation involves non-pathogenic lactic acid bacteria capable of producing 18-carbon 10-hydroxy fatty acids and a yeast capable of oxidizing the 18-carbon hydroxy fatty acids to 12-carbon hydroxy fatty acids via beta-oxidation pathway.

As used herein, “non-pathogenic lactic acid bacteria” are lactic acid bacteria that are not involved in causing disease. Non-limiting examples of non-pathogenic lactic acid bacteria can include species from these genera: Lactobacillus, Leuconostoc, Pediococcus, Lactococcus, and Streptococcus, in addition to some Carnobacterium, Enterococcus, Oenococcus, Tetragenococcus, Vagococcus, and Weisella species. Without wishing to be bound by any theory, in some embodiments, the non-pathogenic lactic acid bacteria have membrane-associated 10-hydratases that can convert unsaturated fatty acids to 10-hydroxy fatty acids. In some embodiments, the non-pathogenic lactic acid bacteria can include Lactobacillus acidophilus, Lactobacillus rhamnosus, Lactobacillus reuteri, Lactobacillus casei, Lactobacillus plantarum, Lactobacillus bulgaricus, Lactobacillus delbrueckii, Lactobacillus helveticus, Lactobacillus paracasei, Lactobacillus johnsonii, and combinations thereof. In some preferred embodiments, the non-pathogenic lactic acid bacteria can include Lactobacillus lactis, Lactobacillus plantarum, Lactobacillus casei, Lactobacillus acidophilus, or combinations thereof.

In some embodiments, the yeast capable of oxidizing 18-carbon hydroxy fatty acids to 12-carbon can include a Yarrowia yeast, a Saccharomyces yeast, a Candida yeast, or combinations thereof. In still other embodiments, the yeast can include Yarrowia lipolytica, Candida petrophilum, or combinations thereof. Without wishing to be bound by any theory, in some embodiments, the yeast can produce lipase to hydrolyze triacylglycerides and can also utilize peroxisomal beta-oxidation to catabolize long-chain fatty acids such as 18-carbon 10-hydroxy fatty acids via oxidation to generate 12-carbon 4-hydroxy fatty acids.

Designing and controlling mixed cultures of bacteria and yeast is notoriously difficult, but these inventors have surprisingly developed a balanced blend of microorganisms that generates useful taste compounds. In some embodiments, a seed culture of the non-pathogenic lactic acid bacteria and/or the yeast are prepared prior to the step of co-culturing the bacteria and yeast together. In some embodiments, the non-pathogenic lactic acid bacteria seed culture is created by selecting bacterial colonies from plates and inoculating them into MRS broth and incubating with agitation at 250 revolutions per minute (rpm) at a temperature of 30 C for 6-24 hours. MRS media are known for their suitability to grow lactic acid bacteria, but any other suitable media could be used as well.

In some embodiments, the yeast seed culture is created by selecting colonies from plates and inoculating them into yeast-extract peptone dextrose (YPD or YEPD) broth and incubating with agitation at 250 rpm at a temperature of 30 C for 6-24 hours. YPD media are known for their suitability to grow yeast, but any other suitable media could be used as well.

For the co-culturing step, in some embodiments, the non-pathogenic lactic acid bacteria seed culture is added to a fermenter containing MRS broth to create a non-pathogenic lactic acid bacterial culture as an initial step in the fermentation. Conventional fermenters are suitable for this step and will be familiar to those of skill in the art. These fermenters allow control of parameters including, but not limited to, pH, agitation, temperature, time, aeration, and the like. One of the many advantages of the inventive processes is the ability to use just one piece of fermenting equipment (i.e. a one-pot process). These simplified processes are faster and give desirable results. In some embodiments, the non-pathogenic lactic acid bacterial culture initial fermentation step has a pH of from about 4 to about 8 and a temperature of from about 25 C to about 40 C for from about 5 hours to about 8 hours. The pH is maintained by adding a base such as sodium hydroxide. Additionally, the dissolved oxygen (DO) content can be maintained at a level of saturation below about 35% saturation by varying the agitation and using an air sparging rate of from about 0.1 to about 1.5 vvm (gas volume flow per unit of liquid volume per minute).

In some embodiments, once no more base is needed to maintain the pH of the non-pathogenic lactic acid bacterial culture, the yeast seed culture is then added to create the mixed culture. In some embodiments, a nutrient feeding stock can be added with the yeast starter culture. This nutrient feeding stock can help the fermentation proceed efficiently as long as the rate of addition does not result in the production of too much acid. Therefore, the precise rate of addition will depend on the size of the fermenter, etc. In some embodiments, the nutrient feeding stock can include a carbon source such as 25%-50% w/w sucrose, an organic nitrogen source such as 5%-15% w/w yeast extract, and an inorganic nitrogen source such as 7%-12% triammonium citrate. In still other embodiments, the nutrient feed stock can be added at a rate of from about 4 to about 12 milliliters/hour/liter of fermentation media. In some embodiments, the temperature of the non-pathogenic lactic acid bacterial culture can be lowered to from about 37 C to about 30 C before adding the yeast starter culture.

In some embodiments, the mixed culture is held in the fermenter for about 15 to about 25 hours before adding the non-hydrolyzed vegetable oil and beginning the fermentation step that involves the biotransformation of non-hydrolyzed vegetable oil into taste compounds. As used herein, “non-hydrolyzed vegetable oil” is understood to mean a triglyceride material with minimal amounts of free fatty acids. In some embodiments, the non-hydrolyzed vegetable oil has less than 2% w/w free fatty acids. In some embodiments, the non-hydrolyzed vegetable oil has at least 75% w/w linoleic acid. In some embodiments, the non-hydrolyzed vegetable oil includes sunflower oil, safflower oil, or combinations thereof. In other embodiments, the non-hydrolyzed vegetable oil includes oils with at least 70% unsaturated fatty acids such as oleic acid, linoleic acid, and linolenic acid. Non-limited examples of such non-hydrolyzed vegetable oils with high levels of unsaturated fatty acids include soy oils, canola oils, avocado oils, and the like.

In some embodiments, the non-hydrolyzed vegetable oil fermentation has a fermentation time (which begins when the non-hydrolyzed vegetable oil is added to mixed culture) of from about 30 hours to about 200 hours. In some embodiments, the fermentation time is from about 30, 35, 40, 45, 48, 50, 55, 60, 65, 70, 75, 80, 85, 90, 96, 100, 105, 110, 115, 120, 125, 130, 135, 140, 145, 150, 155, 160, 165, 170, 175, 180, 185, 190, or 195 to about 40, 45, 48, 50, 55, 60, 65, 70, 75, 80, 85, 90, 96, 100, 105, 110, 115, 120, 125, 130, 135, 140, 145, 150, 155, 160, 165, 170, 175, 180, 185, 190, 195, or 200 hours. In some embodiments, additional non-hydrolyzed vegetable oil can be added at any time during the fermentation. In some embodiments, additional non-hydrolyzed vegetable oil is added at the fermentation time of 5, 8, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 195 hours.

In some embodiments, the non-hydrolyzed vegetable oil fermentation has a fermentation temperature of from about 20 C to about 37 C. In some embodiments, the fermentation temperature is from about 20 C, 21 C, 22 C, 23 C, 24 C, 25 C, 26 C, 27 C, 28 C, 29 C, 30 C, 31 C, 32 C, 33 C, 34 C, 35 C, or 36 C to about 21 C, 22 C, 23 C, 24 C, 25 C, 26 C, 27 C, 28 C, 29 C, 30 C, 31 C, 32 C, 33 C, 34 C, 35 C, 36 C, or 37 C.

In some embodiments, the non-hydrolyzed vegetable oil fermentation has a fermentation pH of from about 4 to about 8. In some embodiments, the fermentation pH is from about 4.0, 4.5, 5.0, 5.5, 5.6, 5.7, 5.8, 5.9, 6.0, 6.1, 6.2, 6.3, 6.4, 6.5, 7.0, or 7.5 to about 4.5, 5.0, 5.5, 5.6, 5.7, 5.8, 5.9, 6.0, 6.1, 6.2, 6.3, 6.4, 6.5, 7.0, 7.5, or 8.0. Those of skill in the art will be familiar with conventional means for pH control including adding bases such as sodium hydroxide and the like.

In some embodiments, the non-hydrolyzed vegetable oil fermentation has a fermentation DO content of less than 35%. In some embodiments, the DO content can be managed by adjusting agitation and air flow rates. In some embodiments, the agitation rate can be from about 300 rpm to about 1000 rpm, and in some embodiments, the air flow rate can be from about 0.1 to about 1 vvm.

As to the acidification step in the method of making taste compounds, in some embodiments, admixing an acid can slow or end the fermentation. In some embodiments, the admixing with acid step results in the mixed culture fermentation medium having an acidification pH of from about 3 to about 4. In other embodiments, the acidification pH is from about 3, 3.1, 3.2, 3.3, 3.4, 3.5, 3.6, 3.7, 3.8, or 3.9 to about 3.1, 3.2, 3.3, 3.4, 3.5, 3.6, 3.7, 3.8, 3.9, or 4. Conventional admixing means can be used for the admixing with acid step and will be familiar to those of skill in the art. These conventional means can include metering pumps, stirring paddles, and the like.

In some embodiments, the acid used in the acidification step can include hydrochloric acid, nitric acid, phosphoric acid, sulfuric acid, citric acid, or combinations thereof.

As to the lactonization step, this heating of the acidified fermentation medium can be accomplished by autoclaving. Conventional means and equipment can be used for the lactonization/autoclaving/heating step and those of skill in the art will be familiar with such means and equipment. Without wishing to be bound by any theory, this autoclaving step can facilitate lactonization reactions due to the temperatures and times involved.

In some embodiments, the lactonization step has a lactonization temperature of from about 90 C to about 130 C. In other embodiments, the autoclaving step has an autoclaving temperature of from about 90 C, 95 C, 100 C, 105 C, 110 C, 115 C, 116 C, 117 C, 118 C, 119 C, 120 C, 121 C, 122 C, 123 C, 124 C, 125 C, 126 C, 127 C, 128 C, or 129 C to about 95 C, 100 C, 105 C, 110 C, 115 C, 116 C, 117 C, 118 C, 119 C, 120 C, 121 C, 122 C, 123 C, 124 C, 125 C, 126 C, 127 C, 128 C, 129 C, or 130 C.

In some embodiments, the lactonization step has lactonization time of from about 10 minutes to about 60 minutes. In other embodiments, the autoclaving time is from about 10, 12, 15, 17, 20, 22, 25, 27, 30, 35, 40, 45, 50, or 55 minutes to about 12, 15, 17, 20, 25, 27, 30, 35, 40, 45, 50, 55, or 60 minutes.

In some embodiments, the method of making taste compounds further comprises a solvent extraction step following the autoclaving step to create a fermented extract. Conventional means can be used for this solvent extraction step and will be familiar to those of skill in the art. In some embodiments, the solvent extraction step involves a solvent such as ethyl acetate, ethanol, hexanes, cyclohexane, or combinations thereof.

In some embodiments, the method of making taste compounds further comprises a distillation step following the solvent extraction step to create a refined lactones product. In some embodiments, the refined lactones product includes at least 70% gamma-dodecen-6-lactone. In other embodiments, the refined lactones product includes from about 5% to about 30% gamma-dodecalactone w/w by weight of the refined lactones product.

In some embodiments, the at least one gamma lactone generated by the method of making taste compounds includes gamma-dodecen-6-lactone, gamma-dodecalactone, or combinations thereof. In some embodiments, the at least one hydroxy fatty acid includes 10-hydroxyoctadecanoic acid, 10-hydroxy-12-octadecenoic acid, or combinations thereof.

In addition to the inventive process of making taste compounds, these inventors have also invented fermented extracts. In some embodiments, the inventions include a fermented extract extracted from a mixed culture fermentation of a non-hydrolyzed vegetable oil. In some embodiments, the mixed culture fermentation comprises at least one bacterium and at least one yeast.

In some embodiments, the at least one bacterium is capable of producing 18-carbon 10-hydroxy fatty acids, and in some embodiments, the at least one bacterium includes a non-pathogenic lactic acid bacterium. In some embodiments, the at least one bacterium can include Lactobacillus lactis, Lactobacillus planatarum, Lactobacillus casei, Lactobacillus acidophilus, or combinations thereof.

In some embodiments, the at least one yeast is capable of oxidizing 18-carbon 10-hydroxy fatty acids to produce 12-carbon 4-hydroxy fatty acids. In some embodiments, the at least one yeast can include Yarrowia, Saccharomyces, Candida, or combinations thereof. In still other embodiments, the at least one yeast includes Yarrowia lipolytica, Candida petrophilum, or combinations thereof.

In some embodiments, the non-hydrolyzed vegetable oil of the fermented extract extracted from a mixed culture fermentation includes sunflower oil, safflower oil, or combinations thereof. In some embodiments, the non-hydrolyzed vegetable oil has at least 75% linoleic acid. In still other embodiments, the non-hydrolyzed vegetable oil has at least 80%, 85%, 90%, or 95% linoleic acid. In some embodiments, the non-hydrolyzed vegetable oil has at least 70% unsaturated fatty acids.

In some embodiments, the fermented extract further comprises at least one 18-carbon 10-hydroxy fatty acid, at least one 12-carbon gamma-lactone, or combinations thereof. In some embodiments, the fermented extract further comprises from about 10% to about 50% w/w gamma-dodecen-6-lactone and from about 5% to about 25% w/w gamma-dodecalactone. In still other embodiments, the fermented extract has a ratio of gamma-dodecen-6-lactone to gamma-dodecalactone of from about 2:1 to about 4:1.

In some embodiments, this fermented extract further comprises a solvent. That solvent can include ethanol, ethyl acetate, or combinations thereof.

Another set of inventions developed by these inventors includes a product comprising a fermented extract and a product base wherein the fermented extract is extracted from a mixed culture fermentation of non-hydrolyzed vegetable oil. The product base can include a full range of both liquid and solid products including, but not limited to, human foods, animal foods, dietary supplements, medicaments, galenical compositions, cosmetic compositions, fragrance compositions, perfumed articles, pharmaceutical compositions, or combinations thereof. In some embodiments, the product base can include beverages such as dairy milks and/or non-dairy milks; alcoholic beverages; frozen desserts such as ice cream, ice milk, ices, sherbets, and the like; as well as cheeses; cheese sauces; salty snacks; granolas and granola bars; or ready-to-eat cereals. In some embodiments, the product base includes a dairy product, a meat product, or a plant-based protein product.

In some embodiments, the fermented extract is present in an olfactory effective amount. As used herein, “olfactory effective amount” is understood to mean an amount that will contribute particular olfactory characteristics, but the flavor, taste, and aroma effect on the overall food product will be the sum of the effects of the ingredients used to make the product. The particular amount imparting an olfactory effect will vary depending on many factors including the relative amounts of the product ingredients and the desired effect of the fermented extract. In some embodiments, the fermented extract is present in an amount of from about 10 ppb to about 1000 ppm. In some embodiments, the fermented extract is present in an amount of from about 10 ppb, 20 ppb, 30 ppb, 40 ppb, 50 ppb, 60 ppb, 70 ppb, 80 ppb, 90 ppb, 100 ppb, 110 ppb, 120 ppb, 130 ppb, 140 ppb, 150 ppb, 160 ppb, 170 ppb, 180 ppb, 190 ppb, 200 ppb, 210 ppb, 220 ppb, 230 ppb, 240 ppb, 250 ppb, 260 ppb, 270 ppb, 280 ppb, 290 ppb, 300 ppb, 310 ppb, 320 ppb, 330 ppb, 340 ppb, 350 ppb, 360 ppb, 370 ppb, 380 ppb 390 ppb, 400 ppb, 410 ppb, 420 ppb, 430 ppb, 440 ppb, 450 ppb, 460 ppb, 470 ppb, 480 ppb, or 490 ppb to about 20 ppb, 30 ppb, 40 ppb, 50 ppb, 60 ppb, 70 ppb, 80 ppb, 90 ppb, 100 ppb, 110 ppb, 120 ppb, 130 ppb, 140 ppb, 150 ppb, 160 ppb, 170 ppb, 180 ppb, 190 ppb, 200 ppb, 210 ppb, 220 ppb, 230 ppb, 240 ppb, 250 ppb, 260 ppb, 270 ppb, 280 ppb, 290 ppb, 300 ppb, 310 ppb, 320 ppb, 330 ppb, 340 ppb, 350 ppb, 360 ppb, 370 ppb, 380 ppb 390 ppb, 400 ppb, 410 ppb, 420 ppb, 430 ppb, 440 ppb, 450 ppb, 460 ppb, 470 ppb, 480 ppb, 490 ppb, or 500 ppb. In other embodiments, the fermented extract is present in an amount of from about 0.5 ppm, 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, or 9.5 ppm to about 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, 9.5 ppm, or 10 ppm. In still other embodiments, the fermented extract is present in an amount of from about 10 ppm, 20 ppm, 30 ppm, 40 ppm, 50 ppm, 60 ppm, 70 ppm, 80 ppm, 90 ppm, 100 ppm, 110 ppm, 120 ppm, 130 ppm, 140 ppm, 150 ppm, 160 ppm, 170 ppm, 180 ppm, or 190 ppm to about 20 ppm, 30 ppm, 40 ppm, 50 ppm, 60 ppm, 70 ppm, 80 ppm, 90 ppm, 100 ppm, 110 ppm, 120 ppm, 130 ppm, 140 ppm, 150 ppm, 160 ppm, 170 ppm, 180 ppm, 190 ppm, or 200 ppm. In some embodiments, the fermented extract is present in an amount of from about 100 ppm, 200 ppm, 300 ppm, 400 ppm, 500 ppm, 600 ppm, 700 ppm, 800 ppm, or 900 ppm to about 200 ppm, 300 ppm, 400 ppm, 500 ppm, 600 ppm, 700 ppm, 800 ppm, 900 ppm, or 1000 ppm.

In some embodiments, the fermented extract is a is a refined lactones product comprising 50% to about 95% w/w gamma-dodecen-6-lactone and from about 5% to about 50% w/w gamma-dodecalactone. In some embodiments, the refined lactones product has a ratio of gamma-dodecen-6-lactone to gamma-dodecalactone of from about 2:1 to about 4:1.

In some embodiments, the refined lactones product is present in an olfactory effective amount. In some embodiments, refined lactones product is present in an amount of from about 10 ppb to about 200 ppm. In some embodiments, the refined lactones product is present in an amount of from about 10 ppb, 20 ppb, 30 ppb, 40 ppb, 50 ppb, 60 ppb, 70 ppb, 80 ppb, 90 ppb, 100 ppb, 110 ppb, 120 ppb, 130 ppb, 140 ppb, 150 ppb, 160 ppb, 170 ppb, 180 ppb, 190 ppb, 200 ppb, 210 ppb, 220 ppb, 230 ppb, 240 ppb, 250 ppb, 260 ppb, 270 ppb, 280 ppb, 290 ppb, 300 ppb, 310 ppb, 320 ppb, 330 ppb, 340 ppb, 350 ppb, 360 ppb, 370 ppb, 380 ppb 390 ppb, 400 ppb, 410 ppb, 420 ppb, 430 ppb, 440 ppb, 450 ppb, 460 ppb, 470 ppb, 480 ppb, or 490 ppb to about 20 ppb, 30 ppb, 40 ppb, 50 ppb, 60 ppb, 70 ppb, 80 ppb, 90 ppb, 100 ppb, 110 ppb, 120 ppb, 130 ppb, 140 ppb, 150 ppb, 160 ppb, 170 ppb, 180 ppb, 190 ppb, 200 ppb, 210 ppb, 220 ppb, 230 ppb, 240 ppb, 250 ppb, 260 ppb, 270 ppb, 280 ppb, 290 ppb, 300 ppb, 310 ppb, 320 ppb, 330 ppb, 340 ppb, 350 ppb, 360 ppb, 370 ppb, 380 ppb 390 ppb, 400 ppb, 410 ppb, 420 ppb, 430 ppb, 440 ppb, 450 ppb, 460 ppb, 470 ppb, 480 ppb, 490 ppb, or 500 ppb. In other embodiments, the refined lactones product is present in an amount of from about 0.5 ppm, 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, or 9.5 ppm to about 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, 9.5 ppm, or 10 ppm. In still other embodiments, the refined lactones product is present in an amount of from about 10 ppm, 20 ppm, 30 ppm, 40 ppm, 50 ppm, 60 ppm, 70 ppm, 80 ppm, 90 ppm, 100 ppm, 110 ppm, 120 ppm, 130 ppm, 140 ppm, 150 ppm, 160 ppm, 170 ppm, 180 ppm, or 190 ppm to about 20 ppm, 30 ppm, 40 ppm, 50 ppm, 60 ppm, 70 ppm, 80 ppm, 90 ppm, 100 ppm, 110 ppm, 120 ppm, 130 ppm, 140 ppm, 150 ppm, 160 ppm, 170 ppm, 180 ppm, 190 ppm, or 200 ppm.

Contributions to olfactory characteristics can include the diminishment of less desirable characteristics, the addition of desirable characteristics, the enhancement of desirable characteristics, or combinations thereof. These olfactory characteristic contributions can be measured by sensory testing. Those of skill in the art will be familiar with various sensory testing methodologies, and any conventional method can be used for measurement of the inventive compositions described here. For example, a trained panel of experts can use numerical line scores of zero to five or zero to ten or zero to fifteen to indicate the perceived level of an olfactory characteristic. Alternatively, various quantitative methodologies are available such as quantitative descriptive analysis. In some embodiments, adding the fermented extract and/or refined lactones product can mask or decrease/limit the perception of an off-odor or off-taste in a product base. For example, a number of plant-based foods (both milk-type beverages and meat-type products) can have off tastes that have been described as “beany” or the like. In some embodiments, the fermented extract and/or refined lactones product can decrease the perception of off-notes as compared to a product base without the fermented extract when measured by sensory testing.

In embodiments in which the fermented extract contributes or enhances desirable characteristics, the product has an increased perception of at least one sensory attribute as compared to a product base without the fermented extract as measured by sensory testing.

In some embodiments, the fermented extract can contribute dairy-like characteristics to the product. In some of those embodiments, the product has an increased perception of dairy notes when compared to a product base without the fermented extracted when measured by sensory testing.

In some embodiments, the product has an increased perception of sensory attributes such as creamy, milky, fatty, waxy, rich, sweet, peachy, lactonic, fruity, or combinations thereof.

These inventors have also invented fermentation media. In some embodiments, a fermentation medium comprises at least one bacterium, at least one yeast, and at least one non-hydrogenated vegetable oil. In some embodiments, the at least one bacterium can be capable of producing 18-carbon 10-hydroxy fatty acids, and in some embodiments, the at least one bacterium can be a non-pathogenic lactic acid bacterium. In some embodiments, suitable bacteria can include Lactobacillus lactis, Lactobacillus plantarum, Lactobacillus casei, Lactobacillus acidophilus, or combinations thereof.

In some embodiments, the at least one yeast of the fermentation medium can be capable of oxidizing 18-carbon 10-hydroxy fatty acids to product 12-carbon 4-hydroxy fatty acids. In some embodiments, the at least one yeast includes Yarrowia, Saccharomyces, Candida, or combinations thereof. In still other embodiments, the at least one yeast includes Yarrowia lipolytica, Candida petrophilum, or combinations thereof.

In some embodiments, the non-hydrolyzed vegetable oil of the fermentation medium includes sunflower oil, safflower oil, or combinations thereof. In other embodiments, the non-hydrolyzed vegetable oil contains at least 70% unsaturated fatty acids. And in still other embodiments, the non-hydrolyzed vegetable oil has at least 75% linoleic acid.

In addition to the other inventions described here, the inventors have further invented a method of modifying a product base. In some embodiments, a method of modifying a product base includes the steps of adding an olfactory effective amount of a fermented extract extracted from a mixed culture fermentation of non-hydrolyzed vegetable oil to a product base to create a modified product. Non-limiting examples of product bases can include human foods, animal foods, dietary supplements, medicaments, galenical compositions, cosmetic compositions, fragrance compositions, perfumed articles, or pharmaceutical compositions. In some embodiments, the product base includes a dairy product, a meat product, a plant-based product, or combinations thereof. Conventional means can be used for adding the fermented extract, and those of skill in the art will be familiar with typical processes such as metered dosing apparatus, blenders, paddle mixers, and the like.

In some embodiments, the olfactory effective amount of fermented extract is 10 ppb to about 200 ppm. Similar to the description above related to products, the modification of the modified product can include a diminishment of less desirable characteristics, an addition of desirable characteristics, an enhancement of desirable characteristics, or combinations thereof. And these modifications can be measured by sensory testing. In some embodiments, the modified product has a decreased perception of off-notes when compared to the product base as measured by sensory testing. In other embodiments, the modified product has an increased perception of dairy notes when compared to the product base as measured by sensory testing. And in still other embodiments, the modified product has an increased perception of at least one sensory attribute wherein the sensory attribute can include creamy, milky, fatty, waxy, rich, sweet, peachy, lactonic, fruity, or combinations thereof when compared to the product base as measured by sensory testing.

The following are provided as specific embodiments of the present invention. Other modifications of this invention will be readily apparent to those skilled in the art. Such modifications are understood to be within the scope of this invention. As used herein, all percentages are weight percent unless otherwise noted, ppm is understood to stand for parts per million, L or 1 is understood to be liter, mL is understood to be milliliter, g is understood to be gram, Kg is understood to be kilogram, mol is understood to be mole, mmol is understood to be millimole, psig is understood to be pound-force per square inch gauge, and mmHg is understood to be millimeters (mm) of mercury (Hg). IFF as used in the examples is understood to mean International Flavors & Fragrances Inc., New York, NY, USA.

### Example 1: Production of Gamma-Dodecen-6-Laconte (FEMA #3780) and Gamma-Dodecalactone (FEMA #2400) for a Biotransformation Time of 48 Hours

To produce taste compounds using a mixed culture of food-grade, non-pathogenic microorganisms, the following actions were taken:
1. Inoculum (seed culture) preparation
   (1) An MRS medium composition was blended together with these ingredients:

| Ingredients | Concentration (g/L) |
|---|---|
| Peptone | 10.0 |
| Meat extract | 8.0 |
| Yeast extract | 4.0 |
| D(+)-glucose | 20.0 |
| Dipotassium hydrogen phosphate | 2.0 |
| Sodium acetate trihydrate | 5.0 |
| Triammonium citrate | 2.0 |
| Magnesium sulfate heptahydrate | 0.2 |
| Manganese sulfate tetrahydrate | 0.05 |
| Tween-80 | 1.0 |

(2) A corresponding YPD medium composition was blended together with these ingredients:
   10 g/L yeast extract, 20 g/L peptone, and 20 g/L glucose

To prepare the seed culture of Lactobacillus plantarum, pure colonies were selected from a plate and inoculated into 100 mL of the MRS medium from above contained in a 500-mL flask, and the flask was shaken at 250 rpm and 30 C overnight. The next day, the L. plantarum seed culture had an OD600 value of 12-13 and was then ready for inoculation.

To prepare the seed culture of Yarrowia lipolytica, pure colonies were selected from a plate and inoculated into 100 mL of the YPD medium shown above contained in a 500-mL flask, and the flask was shaken at 250 rpm and 30 C overnight. The next morning, the Y. lipolytica seed culture had an OD600 value of 35-40 and was then ready for inoculation.
2. Production process
   (1) Culturing of lactic acid bacteria
      A 2-L fermenter was filled with 1 L MRS medium and sterilized at 121 C for 20 minutes. Into the 2-L fermenter, 40 ml (4%) L. plantarum seed culture was added, and the initial culturing parameters shown below were used:
      Temperature: 37 C
      pH: 6.2, as controlled by addition of 5 N NaOH DO: 30% saturation, as maintained by a cascade control with varying agitation speeds as the primary and varying air flow rates of 0.2 to 0.6 vvm as the secondary.
      When glucose was completely consumed by the bacterial culture (usually 6-7 hours), as indicated by no more addition of NaOH, the temperature setting was reduced to 30 C. Into the fermenter, 2 mL 1 M MgSO4, 2 mL 0.1 M MnSO4, and 3 mL oleic acid were added.
   (2) Co-culturing of yeast
      Co-culturing of yeast was started by adding 40 mL (4%) Y. lipolytica seed culture into the fermenter. A nutrient feeding stock containing 35% sucrose, 3% yeast extract, and 10% triammonium citrate was also added into the fermenter at a rate of 6 mL/h for 24 hours. creating the mixed or consortia culture.
   (3) Biotransformation
      After about 16 to 18 hours (overnight) of co-culturing mixed yeast with the bacteria culture, 25 mL high-linoleic acid safflower oil (80%) was added. This is referred to as time 0 of biotransformation. Agitation was set at 500 rpm and air flow rate at 0.3 vvm. The pH was maintained at 6.2 by adding 5N NaOH or 5 N H2SO4. At 8 hr. of biotransformation, additional 25 mL high-linoleic acid safflower oil was added. The biotransformation was terminated at 48 hours by acidifying the medium to pH 3 to 4 using 5 N H2SO4.
   (4) Lactonization, extraction, and purification
      The acidified medium was autoclaved at 121 C for 20 min, which facilitates the lactonization reaction. Lactones were recovered by extraction with a suitable organic solvent (for example, ethyl acetate). The organic phase was then decanted and ethyl acetate solvent removed by evaporation. The resultant flavor oil was distilled under partial vacuum (1 millibar) and a fraction distilled between 95-100 C was collected to afford a refined product comprising about 78% gamma-dodecen-6-lactone and 22% gamma-dodecalactone.
3. Results
   Table 1 shows the production of two major gamma-lactones from safflower oil by this consortia culture of Lactobacillus plantarum and Yarrowia lipolytica over a biotransformation time of 48 hours.

TABLE 1
Consortia culture production of gamma-dodecen-6-lactone
and gamma-dodecalactone from safflower oil over time

| Biotransformation | Gamma-dodecen-6-lactone | Gamma-dodecalactone |
|---|---|---|
| time (hours) | Titer (g/L) | Titer (g/L) |
| 0 (safflower | | |
| oil added) | | |
| 8 (safflower | 0.08 | 0.05 |
| oil added) | | |
| 22 | 0.8 | 0.21 |
| 28 | 0.78 | 0.2 |
| 30 | 0.85 | 0.22 |
| 48 | 1.4 | 0.38 |

Table 2 shows the changes in free fatty acids as a result of the action of lipases from Yarrowia and consumption by the consortia culture of Lactobacillus and Yarrowia over a biotransformation time of 48 hours.

TABLE 2
Consortia culture production of fatty
acids from safflower oil over time

| Biotransformation | Stearic Acid | Oleic Acid | Linoleic Acid |
|---|---|---|---|
| time | Titer (g/L) | Titer (g/L) | Titer (g/L) |
| 0 (safflower | | | |
| oil added) | | | |
| 8 (safflower | 1 | 9 | 15 |
| oil added) | | | |
| 22 | 2 | 9 | 23 |
| 28 | 1 | 5 | 12 |
| 30 | 1.2 | 4 | 10 |
| 48 | 0.8 | 3 | 3.5 |

### Example 2: Production of Gamma-Dodecen-6-Lactone (FEMA #3780) and Gamma-Dodecalactone (FEMA #2400) for a Biotransformation Time of 96 Hours

The production procedures for gamma-dodecen-6-lactone and gamma-dodecalactone were the same as described in Example 1 except that the biotransformation time was extended to 96 hours with an air flow rate of 0.5 vvm, and the amount of safflower oil added at Time 0 and 8 hours was 50 mL each instead of 25 mL.

Table 3 shows the production of the two major gamma-lactones from safflower oil by a consortia culture of Lactobacillus plantarum and Yarrowia lipolytica over a biotransformation time of 96 hours.

TABLE 3
Production of gamma-dodecen-6-lactone and gamma-dodecalactone
from safflower oil by a consortia culture of Lactobacillus plantarum
and Yarrowia lipolytica over a period of 96 hours

| Biotransformation | Gamma-dodecalactone | Gamma-dodecen-6-lactone |
|---|---|---|
| time (hours) | Titer (g/L) | Titer (g/L) |
| 0 (safflower | | |
| oil added) | | |
| 5 | 0.7 | 0.8 |
| 8 (safflower | 0.8 | 1.1 |
| oil added) | | |
| 22 | 1.2 | 3 |
| 28 | 1.3 | 3.2 |
| 32 | 1.5 | 3.3 |
| 96 | 3 | 9 |

Lactones were recovered from the autoclaved fermentation medium by extraction with ethyl acetate, as described in Example 1. The organic phase was decanted and then ethyl acetate solvent removed by evaporation, yielding a flavor oil comprising:
Gamma-dodecen-6-lactone (Dairy Lactone) 20.5%
Gamma-dodecalactone 5.2%
10-hydroxy-12-octadecenoic acid 2.3%
10-hydroxyoctadecanoic acid 1.0%
Palmitic acid 5.0%
Stearic acid 2.8%
Oleic acid 8.7%
Linoleic acid 41.9%

### Example 3: Application in Cream Soda

The refined lactones-containing fermentation extract obtained from Example 1 (78% gamma-dodecen-6-lactone and 22% gamma-dodecalactone) was added in a cream soda at 50 parts per billion (ppb). A panel of 10 judges consisting of certified flavorists and flavor scientists all rated the test soda as more creamy and richer.

### Example 4: Application in Plant Milk

The refined lactones-containing fermentation extract obtained according to Example 1 (78% gamma-dodecen-6-lactone and 22% gamma-dodecalactone) was evaluated in a commercial almond milk. The control sample was prepared by adding 0.01% of an IFF milk flavor in the almond milk. The test sample was prepared by adding 300 ppb of the refined lactones-containing product in the control sample. A technical panel of certified flavorists rated the test sample as creamier, natural tasting, more dairy mouthfeel, lingering, and mouth filling.

### Example 5: Masking Effect on Plant Proteins

The refined lactones-containing fermentation extract obtained according to Example 1 (78% gamma-dodecen-6-lactone and 22% gamma-dodecalactone) was evaluated in a pea protein solution. The base sample was prepared by dispersing pea protein isolate powder (1%) in water. The test sample was prepared by adding 25 ppb of the refined lactones-containing product in the base sample. Descriptive sensory test was conducted on pre-selected attributes. Sample pairs were presented in a blind and random order to a sensory panel. Panelists were instructed to score the attribute intensity by placing a mark on a line scale. As shown in Table 4, beany aroma and beany flavor were significantly reduced by the refined lactone product.

TABLE 4
Descriptive analysis results showing taste masking
effect of fermented extract on pea protein

| | | Protein Base + 25 ppb Refined |
|---|---|---|
| Attribute | 1% Protein Base | Fermented Extract |
| Total Aroma | 12 | 14 |
| Beany Aroma* | 11 | 8 |
| Total Flavor | 12 | 12.5 |
| Beany Flavor* | 11 | 7.5 |
| Bitterness | 4.2 | 4.2 |
| Astringency | 7.5 | 7.5 |

*Indicates a significant difference at p = 0.05

### Example 6: Detection Threshold Determination in Plant Milk

Commercial almond milk was used as the base. The refined lactones-containing fermentation extract prepared according to Example 1 (78% gamma-dodecen-6-lactone and 22% gamma-dodecalactone) was added to the base at various concentrations, ranging from 10 ppb to 120 ppb. Sensory panelists received three coded samples in a blind and random order. Two of the samples were the same and one was different. Panelists were asked to identify the odd sample. Table 5 shows the percent of correct response from panelists as a function of dose concentrations in almond milk. The detection threshold was estimated to be about 50 ppb in almond milk when the detection threshold is defined as the concentration at which detection occurs 50% of the time.

TABLE 5
Dose response plot for detection threshold
determination in almond milk

| Concentration (ppb) | % Correct Response |
|---|---|
| 10 | 28 |
| 55 | 52 |
| 100 | 85 |
| 120 | 100 |

### Example 7: Determination of Upper Limit for Use in Plant Milk

A series of test samples containing varying amounts of the lactones-containing refined fermentation extract obtained according to example 1 (78% gamma-dodecen-6-lactone and 22% gamma-dodecalactone), were prepared in an almond milk base, ranging from 0.2 ppm, 10 ppm, 50 ppm, 100 ppm, 150 ppm, and 200 ppm. A technical panel of eight judges tasted the sample series from the lowest to the highest, and selected the samples that were unbearable to taste due to undesirable attributes. As a result, all judges selected the sample containing 200 ppm as unbearable to taste due to the very strong and overpowering aromas imparted by the product. Thus, 200 ppm was determined to be the upper limit for use in plant milk.

### Example 8: Application of Fermented Extract in Plant Milk

The fermented extract prepared according to Example 2 (gamma-dodecen-6-lactone 20.5% and gamma-dodecalactone 5.2%) was evaluated at 300 ppb in an almond milk base. A technical panel of eight judges all rated the test sample as creamier and more mouth filling without any off flavors.

What is claimed is:

1. A fermented extract extracted from a mixed culture fermentation of a non-hydrolyzed vegetable oil.

2. The composition as in claim 1, wherein the mixed culture fermentation comprises at least one bacterium and at least one yeast.

3. The composition as in claim 2, wherein the at least one bacterium includes Lactobacillus lactis, Lactobacillus planatarum, Lactobacillus casei, Lactobacillus acidophilus, or combinations thereof.

4. The composition as in claim 2, wherein the at least one yeast includes Yarrowia lipolytica, Candida petrophilum, or combinations thereof.

5. The composition as in claim 1, wherein the fermented extract comprises from about 10% to about 50% w/w gamma-dodecen-6-lactone and from about 5% to about 25% w/w gamma-dodecalactone.

6. A method of making taste compounds comprising the steps of:
a. co-culturing at least one non-pathogenic lactic acid bacterium and at least one yeast to create a mixed culture;
b. fermenting a non-hydrolyzed vegetable oil in the mixed culture to create a mixed culture fermentation medium;
c. acidifying the mixed culture fermentation medium by admixing an acid following the fermentation step to create an acidified mixed culture fermentation medium; and
d. lactonizing the acidified mixed culture fermentation medium by heating the acidified mixed culture fermentation medium; wherein the method generates at least one of a gamma lactone, an hydroxy fatty acid, or combinations thereof.

7. The method as in claim 6, further comprising at least one step of preparing a seed culture of the at least one non-pathogenic lactic acid bacterium, preparing a seed culture of the at least one yeast prior to the co-culturing step, or combinations thereof.

8. The method as in claim 7, wherein the co-culturing step further comprises first culturing the at least one non-pathogenic lactic acid bacterium by adding the at least one non-pathogenic lactic acid bacterium seed culture to a bacterial fermentation medium to generate an at least one non-pathogenic lactic acid bacterium culture.

9. The method as in claim 8, further comprising adding the at least one yeast seed culture to the at least one non-pathogenic lactic acid bacterium culture to create the mixed culture.

10. The method as in claim 6, wherein the non-hydrolyzed vegetable oil includes sunflower oil, safflower oil, or combinations thereof.

11. The method as in claim 6, further comprising a solvent extraction step following lactonization to create a fermented extract.

12. The method as in claim 11, further comprising a distillation step following the solvent extraction to create a refined lactones product.

13. The method as in claim 12, wherein the refined lactones product includes at least 70% gamma-dodecen-6-lactone w/w by weight of the refined lactones product.

14. The method as in claim 6, wherein the at least one gamma lactone includes gamma-dodecen-6-lactone, gamma-dodecalactone, or combinations thereof.

15. The method as in claim 6, wherein the at least one hydroxy fatty acid includes stearic acid, oleic acid, linoleic acid, or combinations thereof.

16. The method as in claim 6, wherein the at least one non-pathogenic lactic acid bacterium includes Lactobacillus lactis, Lactobacillus planatarum, Lactobacillus casei, Lactobacillus acidophilus, or combinations thereof.

17. The method as in claim 6, wherein the at least one yeast includes Yarrowia lipolytica, Candida petrophilum, or combinations thereof.

18. A taste compound made by the process as in claim 6.

19. A product comprising a fermented extract and a product base wherein the fermented extract is extracted from a mixed culture fermentation of a non-hydrolyzed vegetable oil.

20. The product as in claim 19, wherein the fermented extract is present in an olfactory effective amount.
